(19) United States
(12) Patent Application Publication
(10) Pub. No.: US 2021/0395789 A1
(43) Pub. Date: Dec. 23, 2021

(54) Materials and Methods for Producing Cardiolipin-Like Phospholipids

(71) Applicant: Locus IP Company, LLC, Solon, OH (US)

(72) Inventors: Sean FARMER, North Miami Beach, FL (US); Ken ALIBEK, Solon, OH (US); Albina TSKHAY, Solon, OH (US); Alibek MOLDAKOZHAYEV, Solon, OH (US)

(21) Appl. No.: 16/964,994

(22) PCT Filed: Mar. 11, 2020
(86) PCT No.: PCT/US2020/022026

§ 371 Date: Jul. 26, 2020

Related U.S. Application Data
(60) Provisional application No. 62/817,234, filed on Mar. 12, 2019.
(60) Provisional application No. 62/914,083, filed on Oct. 11, 2019.

(51) Int. Cl. C12P 7/64; C12N 1/16

(52) U.S. Cl. CPC C12P 7/6481; C12R 2001/85; C12N 1/16

(57) ABSTRACT

The subject invention provides methods for producing phospholipids using yeasts not previously known to produce high concentrations of such phospholipids. In particular, Wickerhamomyces anomalus is cultivated in a specially-tailored nutrient medium and under cultivation conditions such that the yeast unnaturally produces high concentrations of phospholipids that resemble human cardiolipin and/or precursors thereof in structure and/or function. Yeast culture compositions are also provided, comprising yeast cells, growth medium, and high concentrations of phospholipids.

### CROSS-REFERENCE TO RELATED APPLICATION

This application claims priority to U.S. Provisional Patent Application Nos. 62/817,234, filed Mar. 12, 2019, and 62/914,083, filed Oct. 11, 2019, both of which are incorporated herein by reference in their entirety.

### BACKGROUND OF THE INVENTION

Microorganisms, such as yeast, fungi and bacteria, are important for the production of a wide variety of bio-preparations that are useful in many settings, such as oil production; agriculture; remediation of soils, water and other natural resources; mining; animal feed; waste treatment and disposal; food and beverage preparation and processing; and human health.

Biosurfactants are surfactants produced by living cells. They are amphiphiles, consisting of two parts: a polar (hydrophilic) moiety and non-polar (hydrophobic) group. Due to their amphiphilic structure, biosurfactants reduce the surface and interfacial tensions between the molecules of liquids, solids, and gases.

Additionally, biosurfactants accumulate at interfaces, thus leading to the formation of aggregated micellar structures in solution. The ability of biosurfactants to form pores and destabilize biological membranes permits their use as, e.g., antibacterial and antifungal agents. Furthermore, biosurfactants are biodegradable, have low toxicity, and can be produced using low-cost renewable resources. They can inhibit microbial adhesion to a variety of surfaces, prevent the formation of biofilms, and can have powerful emulsifying and demulsifying properties.

Interest in biosurfactants has been steadily increasing in recent years due to their diversity, environmentally friendly nature, selectivity, performance under extreme conditions, and potential applications in environmental protection and other fields. Combined with the characteristics of low toxicity and biodegradability, biosurfactants can be useful in a variety of settings and industries. Most biosurfactant-producing microorganisms produce biosurfactants in response to the presence of a hydrocarbon source in the growing media. Other media components, such as concentration of minerals and pH, can also affect microbial biosurfactant production significantly.

Microbial biosurfactants are produced by a variety of microorganisms such as bacteria, fungi, and yeasts, including, for example, Starmerella spp. (e.g., S. bombicola), Pseudomonas spp. (e.g., P. aeruginosa, P. putida, P. florescens, P. Tragi, P. syringae); Flavobacterium spp.; Bacillus spp. (e.g., B. subtilis, B. amyloliquefaciens, B. pumillus, B. cereus, B. licheniformis); Wickerhamomyces spp. (e.g., W. anomalus), Candida spp. (e.g., C. albicans, C. rugosa, C. tropicalis, C. lipolytica, C. torulopsis); Saccharomyces (e.g., S. cerevisiae); Pseudozyma spp. (e.g., P. aphidis); Rhodococcus spp. (e.g., R. erythropolis); Ustilago spp.; Arthrobacter spp.; Campylobacter spp.; Cornybacterium spp.; as well as others.

Biosurfactants can be produced by both prokaryotic and eukaryotic cells. Biosurfactants can include, for example, low-molecular-weight glycolipids, cellobiose lipids, lipopeptides, flavolipids, phospholipids, and high-molecular-weight polymers such as lipoproteins, lipopolysaccharide-protein complexes, and/or polysaccharide-protein-fatty acid complexes.

One important type of phospholipid biosurfactant is cardiolipin, also known by the names 1,3-bis(sn-3′-phosphatidyl)-sn-glycerol, diphosphatidylglycerol lipid, glycerophospholipid or Calcutta antigen. The name “cardiolipin” is derived from where it was first discovered—in the cells of animal hearts. Cardiolipin makes up about 20% of the total lipid composition of the inner mitochondrial membrane of animal cells, as well as many plant cells. It can be also be found in membranes of some prokaryotic organisms. For example, most bacterial membranes contain cardiolipin, as well as some yeasts and fungi (e.g., Saccharomyces cerevisiae and Aspergillus fumigatus).

Cardiolipin molecules comprise two phosphatidic acid moieties connected by a glycerol at the center, as well as four distinct acyl groups with fatty acid residues attached thereto. Because of the four acyl groups, cardiolipin species can vary widely in terms of the type and/or types of fatty acids that make up their tails. In general, the head group of cardiolipin and certain amino acid residues interact strongly via electrostatic forces, hydrogen bonds, and water molecules to facilitate, for example, conformational changes to proteins to modulate their structures and functions. The acyl chains, on the other hand, retain their flexibility and interact through van der Waals forces with various proteins and surfaces. Additionally, cardiolipin may modulate the activity of some membrane proteins by forming clusters and non-bilayer structures. (“The LipidWeb” 2018).

Cardiolipin plays a crucial role in mitochondrial activity, such as cristae formation, increase of respiratory super-complexes and ATP synthase efficiency, binding and regulating catalytic activity of super-complexes I, II, III and IV, restriction of proton pumping, maintenance of mitochondrial membrane potential, interaction with cytochrome c, and serving as a mitochondrial signaling platform during regulation of apoptosis, as well as being a ROS primary attack site.

Cardiolipin deficiencies and/or abnormalities in humans are linked to certain clinical maladies related to metabolism and mitochondrial function. These include, for example, Barth's syndrome, Parkinson's disease and Alzheimer's disease, non-alcoholic fatty liver disease, heart failure, Tangier disease, diabetes, antiphospholipid syndrome, and certain cancers. For example, Barth syndrome, which is characterized by development of cardiac and skeletal myopathy, as well as neutropeniais is caused by a mutation in the tazl gene, which encodes the tafazzin enzyme involved in production of cardiolipin.

Biosurfactants have the potential to play highly beneficial roles in, for example, the cosmetics and healthcare industries; however, one limiting factor in commercialization of biosurfactants, has been the expense and difficulties in producing them on a large scale. Thus, more efficient methods are needed for producing the large quantities of microbe-based products, such as cardiolipins and/or cardiolipin-like compounds, that are required for such applications.

### BRIEF SUMMARY OF THE INVENTION

The subject invention provides materials and methods for the efficient production and use of beneficial microbes, as well as for the production, purification and use of substances, such as metabolites, derived from these microbes and the substrate in which they are produced.

In particular, the subject invention provides materials and methods for producing and purifying phospholipids using yeasts. In specific embodiments, the phospholipids are cardiolipin (CL) molecules, precursor molecules thereof, and/or phospholipids having similar structures and/or functions to CL and/or precursors thereof. Advantageously, the subject invention increases efficiency and reduces costs associated with phospholipid production, compared to traditional production methods.

In general, the subject methods involve cultivating a yeast strain under specially-tailored conditions, wherein these conditions influence one or more biological mechanisms, which, when activated in the yeast, result in the unnatural high concentration production of the desired growth by-product(s) (e.g., phospholipids). In certain embodiments, the one or more biological mechanisms are inactive or weakly active in the yeast, absent these influencing conditions. Advantageously, the subject methods do not require the use of genetically-modified organisms.

In specific embodiments, the methods utilize the yeast Wickerhamomyces anomalus, also known as Pichia anomala, W. anomalus was previously not known to possess the biological mechanism(s) and/or capability for producing high concentrations of cardiolipin (CL) molecules, precursor molecules thereof, and/or phospholipids having similar structures and/or functions to CL and/or precursors; thus, the methods of the subject invention provide for the unexpected and advantageous result of non-natural, high concentration production of these phospholipids. Furthermore, in some embodiments, the subject methods lead to production of phospholipid molecules that are surprisingly similar in structure to CL and/or precursors thereof found in human and mammalian cells.

In one embodiment, the method comprises inoculating a customized nutrient medium with an inoculum of a yeast to produce a yeast culture; and cultivating the yeast culture for an amount of time and under conditions that are favorable for production of phospholipids. In certain embodiments, the yeast is W. anomalus, or a species related thereto, such as, for example, Pichia guilliermondii, Pichia occidentalis, and/or Pichia kudriavzevii.

In certain embodiments, the yeast culture is cultivated for an amount of time ranging from about 2 days to about 10 days, or about 3 days to about 9 days. In one embodiment, production of the phospholipid molecule can be observed in as little as 24 hours after the start of cultivation.

In one embodiment, the conditions favorable for production of phospholipids include specific temperature, dissolved oxygen (DO) and pH conditions.

In one embodiment, the favorable temperature is about 25 to 30° C. In one embodiment, the favorable DO levels are about 20% to about 50% of saturation.

In one embodiment, the favorable pH levels are about 3.0 to about 7.0. In certain embodiments, the cultivation pH begins at about 6.0 and is lowered to about 3.5 to 4.0 and stabilized.

In certain embodiments, the pH naturally lowers during the course of cultivation. Thus, in some embodiments, the method can comprise simply stabilizing the pH upon reaching a pH of 3.5 to 4.0.

In certain embodiments, the nutrient medium can comprise sources of proteins, amino acids and/or their derivatives, antioxidants, fatty acids, vitamins, nitrogen, potassium, phosphorous, magnesium, calcium, sodium, carbon and/or other trace elements.

In one embodiment, the nutrient medium comprises one or more inorganic salts, such as, e.g., ammonium sulfate, magnesium sulfate, di-potassium phosphate, monosodium phosphate, and/or potassium phosphate.

In one embodiment, the nutrient medium comprises a source of nitrogen, such as, for example, urea; a source of proteins and/or vitamins, such as, for example, yeast extract; and/or a source of fatty acids, such as, for example, canola oil, sunflower oil, and/or soybean oil.

In one embodiment, the nutrient medium comprises one or more sources of carbon, such as a sugar (e.g., glucose).

In one embodiment, the nutrient medium can be supplemented with one or more of biotin, acetyl L-carnitine, alpha-lipoic acid and/or a sugar alcohol (e.g., inositol). In preferred embodiments, the nutrient medium contains a substantial amount of inositol, for example, about 5 to 20 g/L.

Advantageously, in preferred embodiments, the nutrient medium is optimized such that the yeast is induced to produce phospholipids, unnaturally, at a concentration of, for example, 0.1 g/L to 55.0 g/L of the culture medium, or more.

In certain embodiments, the subject invention provides a customized nutrient medium for producing cardiolipin-like phospholipids from a yeast culture, the nutrient medium comprising ammonium sulfate, magnesium sulfate, di-potassium phosphate, monosodium phosphate, potassium phosphate, inositol, urea, yeast extract, glucose, soybean and/or sunflower oil, and, optionally, one or more of biotin, acetyl L-carnitine, alpha-lipoic acid, and trace elements.

In some embodiments, the subject methods can be useful for producing phospholipids that are similar to mammalian and/or human CL and/or precursors thereof. These growth by-products can be retained in the cells of the microorganisms and/or secreted into the solid substrate and/or liquid medium in which the microbes are growing. In certain embodiments, the phospholipids are excreted as extracellular compounds.

In certain embodiments, the methods of the subject invention comprise cultivating a microorganism and/or producing a microbial growth by-product, wherein cultivation is performed using solid state fermentation (SSF), submerged fermentation, or modified versions and/or combinations thereof. Furthermore, the method can comprise aerobic and/or anaerobic fermentation.

The methods can be scaled up or down. Most notably, the methods can be scaled to an industrial scale, i.e., a scale that is suitable for use in supplying phospholipid substances in amounts to meet the demand for commercial applications, for example, production of cosmetics, pharmaceutical compositions and/or supplements for enhancing mitochondrial function.

In certain embodiments, the subject invention provides microbe-based products, as well as their uses in, for example, human health, personal care and cosmetics.

The microbe-based products can comprise the entire culture produced according to the subject methods, including the microorganisms and/or their growth by-products, as well as residual growth medium and/or nutrients. The microorganisms can be live, viable or in an inactive form. They can be in the form of a biofilm, vegetative cells, spores, conidia, hyphae, mycelia and/or a combination thereof. In certain embodiments, no microbes are present, wherein the composition comprises microbial growth by-products, e.g., one or more phospholipids, that have been extracted from the culture and, optionally, purified.

In one embodiment, the microbe-based products can be formulated into a cosmetic product for improving the health and/or appearance of skin. The cosmetic product can be, for example, a face mask, which, when applied to facial skin, can increase the cardiolipin levels of epithelial cells to promote healing of damaged or wounded skin and to restore the skin's youthful appearance.

In one embodiment, the microbe-based products can be formulated into a pharmaceutical composition or supplement for improving mitochondrial function in humans and other animals. The pharmaceutical and/or health supplement can be, for example, an orally-administered composition, or can be formulated for administration by other routes. The product can be used for treating mitochondrial deficiencies and/or secondary mitochondrial dysfunction caused by, for example, mitochondrial myopathy, Leigh syndrome, Barth syndrome, mitochondrial DNA depletion syndrome, Alzheimer's disease, muscular dystrophy, Lou Gehrig's disease, diabetes, cancer and others.

### BRIEF DESCRIPTION OF THE FIGURES

FIG. 1 shows a pathway for biosynthesis of cardiolipin (CL) in eukaryotic mitochondria. Glycero-3-phosphate (G3P) is acylated to lysophosphatidic acid (LPA). LPA is acylated to phosphatidic acid (PA). These steps occur in the outer membrane (OM) of the mitochondria and endoplasmic reticulum. PA then travels into the inner membrane (IM) of the mitochondria, where it is transformed to CDP-diacylglycerol (CDPDG). CDPDG is then converted to phosphatidylglycerophosphate (PGP), which is transformed to phosphatidylglycerol (PG). PG is dimerized into non-mature CL (CLn), also known as monolysocardiolipin (MLCL). The final step is transformation of MLCL to mature unsaturated CL (CLm) by the tafazzin enzyme (Tazl).

FIG. 2 shows Fourier-transform infrared spectroscopy (FTIR) analysis of phospholipids produced according to the subject methods.

FIG. 3 shows P-21 NMR analysis results for a variety of phospholipids. PA=phosphatidic acid; TMP=trimethyl phosphate; PG=phosphatidylglycerol; PE=phosphatidylethanolamine; PC=phosphatidylcholine; PI=phosphatidylinositol; PS=phosphatidylserine; Lyso=having one fatty acid chain removed; SM=sphingomyelin.

FIGS. 4A-4C show (A) H-1 nuclear magnetic resonance (NMR) spectroscopy analysis, (B) C-13 NMR analysis, and (C) P-31 NMR analysis of cardiolipin-like phospholipids produced according to the subject methods.

FIG. 5 shows survival rate of fruit flies fed with a phospholipid according to embodiments of the subject invention compared to control fruit flies.

FIG. 6 shows FT-IR analysis results of the phospholipid product according to embodiments of the subject invention after the culture was treated with ethyl acetate, industrially centrifuged, and the water layer evaporated at 60° C. until 10% water remained.

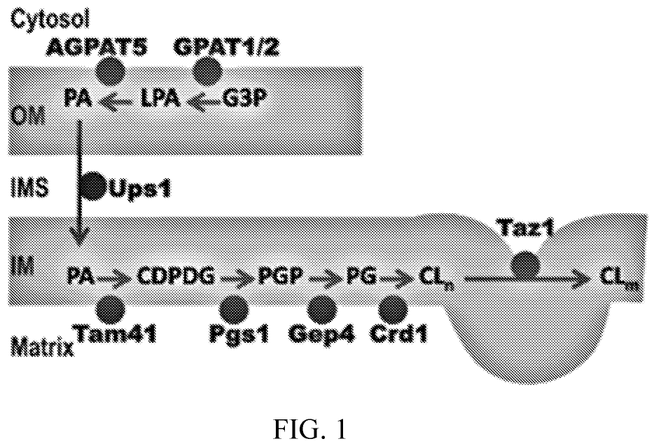

### DETAILED DESCRIPTION

The subject invention provides materials and methods for the efficient production and use of beneficial microbes, as well as for the production and use of substances, such as metabolites, derived from these microbes and the substrate in which they are produced.

In particular, the subject invention provides materials and methods for producing phospholipids, such as CL and/or precursors thereof, in enhanced amounts, and/or substances that closely resemble CL and/or its precursors in structure and function, using yeasts. Advantageously, the subject invention can be scaled for efficient large-scale production of phospholipid molecules for use in, for example, mass production of healthcare, cosmetics and personal care products.

### Selected Definitions

As used herein, a “biofilm” is a complex aggregate of microorganisms, wherein the cells adhere to each other and produce extracellular substances that encase the cells. Biofilms can also adhere to surfaces. The cells in biofilms are physiologically distinct from planktonic cells of the same organism, which are single cells that can float or swim in liquid medium.

As used herein, an “isolated” or “purified” nucleic acid molecule, polynucleotide, polypeptide, protein or organic compound such as a small molecule (e.g., those described below), is substantially free of other compounds, such as cellular material, with which it is associated in nature. A purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. An isolated microbial strain means that the strain is removed from the environment in which it exists in nature. Thus, the isolated strain may exist as, for example, a biologically pure culture, or as spores (or other forms of propagule), optionally, in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 80%, 85%, 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of a metabolite include, but are not limited to, a biosurfactant, enzyme, biopolymer, bioemulsifier, acid, solvent, amino acid, nucleic acid, peptide, protein, lipid, carbohydrate, vitamin and/or mineral.

As used herein, a “microbe-based composition” is a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of propagule, or a mixture of these. The microbes may be planktonic or in a biofilm form, or a mixture of both. The by-products of growth may be, for example, metabolites, cell membrane components, proteins, and/or other cellular components. The microbes may be intact or lysed. In some embodiments, the microbes are present, with medium in which they were grown, in the microbe-based composition. The cells may be present at, for example, a concentration of at least 1×10⁴, 1×10⁵, 1×10⁶, 1×10⁷, 1×10⁸, 1×10⁹, 1×10¹⁰, 1×10¹¹, 1×10¹² or 1×10¹³ or more CFU per gram or milliliter of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise only a portion of the product of cultivation (e.g., only the growth by-products), and/or the microbe-based product may comprise further ingredients that have been added. These additional ingredients can include, for example, stabilizers, buffers, appropriate carriers, such as water, salt solutions, or any other appropriate carrier, added nutrients to support further microbial growth, non-nutrient growth enhancers, such as amino acids, and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, a “precursor” molecule, is a molecule that is involved in the formation of another molecule (e.g., a cardiolipin), and/or a molecule that precedes another molecule in a metabolic pathway.

As used herein “reduction” means a negative alteration, and “increase” means a positive alteration, wherein the negative or positive alteration is at least 1%, 5%, 10%, 25%, 50%, 75%, or 100%.

As used herein, “surfactant” means a surface-active compound that lower the surface tension (or interfacial tension) between two liquids or between a liquid and a solid. Surfactants act as, e.g., detergents, wetting agents, emulsifiers, foaming agents, and dispersants. A “biosurfactant” is a surface-active substance produced by a living cell.

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19, or 20 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially” of the recited component(s).

Unless specifically stated or obvious from context, as used herein, the term “or” is understood to be inclusive. Unless specifically stated or obvious from context, as used herein, the terms “a,” “an,” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims. All references cited herein are hereby incorporated by reference.

### Methods for Producing Phospholipids

The subject invention provides methods for cultivation of microorganisms and production of microbial metabolites and/or other by-products of microbial growth using solid state fermentation, submerged fermentation, or a combination thereof. As used herein “fermentation” refers to growth of cells under controlled conditions. The growth could be aerobic or anaerobic.

In one embodiment, the subject invention provides materials and methods for the production of biomass (e.g., viable cellular material), extracellular metabolites (e.g. small molecules, polymers and proteins), residual nutrients and/or intracellular components (e.g. enzymes and other proteins).

In particular, the subject invention provides materials and methods for producing phospholipids in enhanced amounts, wherein the phospholipids are cardiolipins (CL), precursor molecules to CL, and/or other phospholipid molecules that structurally and/or functionally resemble CL and/or precursors thereof. In some embodiments, the phospholipids are similar in structure and/or function to CL and/or precursors thereof present in human and/or mammalian cells.

Advantageously, the subject invention can be scaled for efficient large-scale production of these phospholipids for use in, for example, mass production of health supplements, cosmetics and personal care products.

In preferred embodiments, the phospholipids produced according to the subject invention have a structure comprising a polar head consisting of at least one phosphatidic acid (PA) molecule. In some embodiments, the PA is bonded to glycerol.

In certain embodiments, the phospholipids produced according to the subject invention can include any phospholipid having one of the following structures:

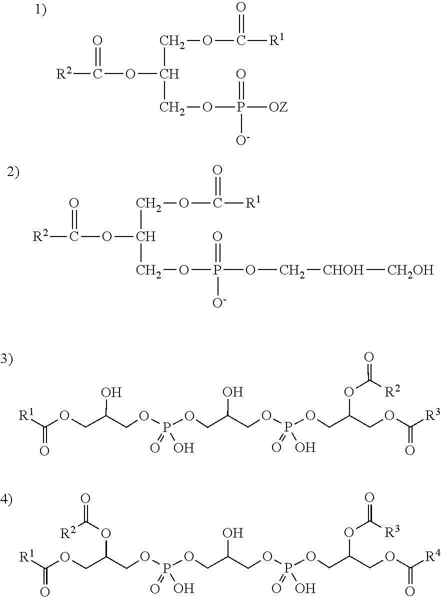

where Z is H, a serine group, choline group, ethanolamine group, inositol group, or glycerol group (see General Formula 2), and

where R1-R4 are the same or different fatty acid side chains having between 14 and 22 carbon atoms and between 0 and 6 double-bonded carbon atoms.

In some embodiments, R1-R4 can be a linoleoyl group (C18:2); an oleoyl group (C18:1); a stearoyl group (C18:0); a margaric group (C17:0); a palmitoleoyl group (C16:1); a palmitoyl group (C16:0); a myristoyl group (C14:0); a docosehexaenoyl group (C22:6); a linolenoyl group (C18:3); an eicosapentaenoyl group (C20:5); and/or a monoenoic fatty acid.

In certain embodiments, the phospholipid has a structure according to General Formula 1) (e.g., a glycerophospholipid). Glycerophospholipids comprise one or more fatty acid lipid group(s), a glycerol backbone, and a phosphate ester. Examples of glycerophospholipids include, but are not limited to, phosphatidic acid, lyso-phosphatidic acid, phosphatidylglycerol, diphosphatidylglycerol, phosphatidylglycerophosphate, phosphatidylglycerophosphoglycerol, Bis(monoacylglycero)phosphate (BMP), Bis(diacylglycero)phosphate (BDP), acylphosphatidylglycerol and phosphatidylethanolamine.

In some embodiments, the glycerophospholipid is a precursor molecule in the formation of CL, where the joining of two glycerophospholipid moieties with a glycerol backbone forms the dimeric structure of CL.

In certain specific embodiments, wherein Z═H, the glycerophospholipid is a phosphatidic acid (PA). In certain specific embodiments, wherein Z=an ethanolamine group, the glycerophospholipid is a phosphatidylethanolamine (PE). In certain specific embodiments, wherein Z=a glycerol group, the glycerophospholipid is a phosphatidylglycerol (PG) (General Formula 2).

In some embodiments, PG and CL both can function as mitochondrial protein stabilizers. In some embodiments, PG triggers CL production. Thus, in some embodiments, PG can be used in place of, or in addition to, CL in certain applications.

In certain specific embodiments, the phospholipid has a structure according to General Formula 3) (e.g., a monolysocardiolipin (MLCL)). In some embodiments, MLCL is a precursor molecule in the formation of CL, where the addition of a fourth fatty acid chain to the MLCL results in a CL.

In certain specific embodiments, the phospholipid has a structure according to General Formula 4) (e.g., a cardiolipin). In certain specific embodiments, the CL comprises four linoleoyl R groups (e.g., tetralinoleoyl CL). Tetralinoleoyl CL is one of over 100 CL molecular species present in brain mitochondria.

In one embodiment, the phospholipid is a structural analogue of CL, and/or a precursor thereof, found in animals and/or plants. As used herein, a “structural analogue” is a compound having a chemical structure similar to that of another compound, but differing from it in respect to a certain component, such as one or more atoms, functional groups, or substructures. Structural analogs may have different physical, chemical, biochemical or pharmacological properties from the molecule of interest.

In some embodiments, the structural analog is D-glucopyranosylcardiolipin, D-alanylcardiolipin, L-lysylcardiolipin, glucosylcardiolipin, or glycocardiolipin.

In one embodiment, the phospholipid can include all stereoisomers and/or constitutional isomers of CL and/or precursors thereof. As used herein, an “isomer” refers to a molecule with an identical chemical formula to another molecule, but having unique structures. Isomers can be constitutional isomers, where atoms and functional groups are bonded at different locations, and stereoisomers (spatial isomers), where the bond structure is the same but the geometrical positioning of atoms and functional groups in space is different.

In certain embodiments, the function of the phospholipids produced according to the subject invention is similar to that of human and/or bovine CL and/or precursors thereof. In some embodiments, this is tested by applying the phospholipid to human skin and observing the effects of the phospholipid for, e.g., promoting the healing of damaged or wounded skin, reducing the appearance of wrinkles, and restoring overall youthful appearance of skin.

In general, the subject methods involve cultivating a yeast strain under specially-tailored conditions, wherein these conditions influence one or more biological mechanisms, which, when activated in the yeast, result in unnatural production of high concentrations of a desired growth by-product(s) (e.g., a phospholipid). In certain embodiments, the one or more biological mechanisms are inactive or weakly active in the yeast, absent these influencing conditions.

In certain embodiments, cultivation is performed using solid state fermentation (SSF), submerged fermentation, or modified versions and/or combinations thereof. Furthermore, the method can comprise aerobic and/or anaerobic fermentation.

The methods can be scaled up or down. Most notably, the methods can be scaled to an industrial scale, i.e., a scale that is suitable for use in supplying phospholipids in amounts to meet the demand for commercial applications, for example, production of cosmetics and/or health supplements.

The microorganisms utilized according to the subject invention can be natural, or genetically modified microorganisms. For example, the microorganisms may be transformed with specific genes to exhibit specific characteristics. In preferred embodiments, however, the microorganism is not genetically modified.

The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In specific embodiments, the methods utilize the yeast Wickerhamomyces anomalus, also known as Pichia anomala. W. anomalus was previously not known to possess the biological mechanism(s) and/or capability for producing phospholipids having CL-like properties; thus, the methods of the subject invention provide for the unexpected and advantageous result of non-natural production of these molecules at high concentrations.

In some embodiments, other yeasts and/or fungi can be utilized according to the subject methods. Yeast and fungus species suitable for use according to the current invention, include Aspergillus spp, Aureobasidium (e.g., A. pullulans), Blakeslea, Candida (e.g., C. apicola, C. bombicola, C. nodaensis), Cryptococcus, Debaryomyces (e.g., D. hansenii), Entomophthora, Hanseniaspora, (e.g., H. uvarum), Hansenula, Issatchenkia, Kluyveromyces (e.g., K. phaffii), Meyerozyma spp. (e.g., M guilliermondii), Mortierella, Mycorrhiza, Phycomyces, Pichia (e.g., P. guilliermondii, P. occidentalis, P. kudriavzevii), Pseudozyma (e.g., P. aphidis), Saccharomyces (e.g., S. boulardii, S. cerevisiae, S. torula), Starmerella (e.g., S. bombicola), Torulopsis, Trichoderma (e.g., T. reesei, T. harzianum, T. hamatum, T. viride), Williopsis (e.g., W. mrakii), Zygosaccharomyces (e.g., Z. bailii), and others. Advantageously, the subject methods do not require the use of genetically-modified organisms.

In one embodiment, the method comprises inoculating a customized nutrient medium with an inoculum of a yeast to produce a yeast culture; and cultivating the yeast culture for an amount of time and under conditions that are favorable for production of phospholipids. In certain embodiments, the yeast is W. anomalus or a species related thereto, such as, for example, Meyerozyma (Pichia) guilliermondii, Pichia occidentalis or Pichia kudriavzevii.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, a daily sample may be taken from the vessel and subjected to enumeration by techniques known in the art, such as dilution plating technique.

In certain embodiments of the subject methods, the customized nutrient medium comprises sources of proteins, amino acids and/or their derivatives, antioxidants, fatty acids, vitamins, nitrogen, potassium, phosphorous, magnesium, calcium, sodium, carbon and/or other trace elements.

In one embodiment, the customized nutrient medium comprises one or more inorganic salts, such as, e.g., potassium dihydrogen phosphate, dipotassium hydrogen phosphate, disodium hydrogen phosphate, magnesium sulfate, magnesium chloride, iron sulfate (e.g., ferrous sulfate heptahydrate), iron chloride, manganese sulfate, manganese sulfate monohydrate, manganese chloride, zinc sulfate, lead chloride, copper sulfate, calcium chloride, calcium carbonate, and/or sodium carbonate. These inorganic salts may be used independently or in a combination of two or more.

In a specific embodiment, the inorganic salts are selected from ammonium sulfate, magnesium sulfate, di-potassium phosphate, monosodium phosphate, and/or potassium phosphate. The inorganic salt(s) can each be added at concentrations of about, for example, 0.1 g/L to 5 g/L, or 0.2 g/L to 4 g/L, or 0.3 g/L to 3 g/L, or 1 g/L to 2.5 g/L.

In one embodiment, the customized nutrient medium comprises one or more sources of nitrogen, such as, for example, potassium nitrate, ammonium nitrate ammonium sulfate, ammonium phosphate, ammonia, urea, and/or ammonium chloride. These nitrogen sources may be used independently or in a combination of two or more.

In a specific embodiment, the nitrogen source(s) comprise urea at a concentration of about, for example, 0.25 g/L to 3.0 g/L, or 0.5 g/L to 1.0 g/L.

In one embodiment, the customized nutrient medium comprises a source of proteins and/or vitamins, such as, for example, yeast extract at a concentration of about, for example, 0.25 g/L to 3.0 g/L, or 0.5 g/L to 1.0 g/L.

In one embodiment, the customized nutrient medium comprises one or more sources of fatty acids, such as, for example, canola oil, sunflower oil, and/or soybean oil at a concentration of about, for example, 20 ml/L to 60 ml/L, or 25 ml/L to 55 ml/L, or 30 ml/L to 50 ml/L.

In one embodiment, the customized nutrient medium comprises one or more sources of carbon, such as carbohydrates, e.g., glucose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as soybean oil, coconut oil, canola oil, rapeseed oil, safflower oil, rice bran oil, olive oil, corn oil, sesame oil, and/or linseed oil; etc. These carbon sources may be used independently or in a combination of two or more.

In a specific embodiment, the carbon source is a carbohydrate, such as glucose, at a concentration of, for example, 10 g/L to 50 g/L, or 15 g/L to 40 g/L, or 20 g/L to 30 g/L.

In one embodiment, the customized nutrient medium can comprise one or more growth factors and/or trace nutrients. Furthermore, sources of vitamins, essential amino acids, and microelements can be included, for example, in the form of flours or meals, such as corn flour, or in the form of extracts, such as potato extract, beef extract, soybean extract, banana peel extract, and the like, or in purified forms. Amino acids such as, for example, those useful for biosynthesis of proteins, can also be included.

Inorganic nutrients, including trace elements such as iron, zinc, copper, manganese, molybdenum and/or cobalt may also be included in the medium at a concentration of, for example. 0.1 ml/L to 10 ml/L, 0.25 ml/L to 5 ml/L or 0.5 ml/L to 2.5 ml/L.

In a specific embodiment, the customized nutrient medium can comprise one or more supplemental additives for inducing increased production of the phospholipids according to the subject invention. These additives can include one or more of biotin, acetyl L-carnitine, alpha-lipoic acid, and/or a sugar alcohol (e.g., inositol, erythritol). Each of these supplemental ingredients can be added at a concentration of about, for example, 0.1 g/L to 20 g/L, or about 0.2 g/L to about 15 g/L, or about 0.5 g/L to about 10 g/L.

In a specific embodiment, the nutrient medium comprises a substantial amount of inositol, for example, from about 5 g/L to 20 g/L, about 8 g/L to 12 g/L, or about 10 g/L

In one exemplary embodiment, the customized nutrient medium comprises ammonium sulfate, magnesium sulfate, di-potassium phosphate, monosodium phosphate, potassium phosphate, biotin, acetyl L-carnitine, alpha-lipoic acid, inositol, urea, yeast extract, glucose, soybean and/or sunflower oil, and trace elements.

In one exemplary embodiment, the customized nutrient medium comprises ammonium sulfate, magnesium sulfate, di-potassium phosphate, monosodium phosphate, potassium phosphate, inositol, urea, yeast extract, glucose, soybean and/or sunflower oil, and, optionally, one or more of trace elements, biotin, acetyl L-carnitine and alpha-lipoic acid.

Advantageously, in preferred embodiments, the nutrient medium is optimized such that the yeast is influenced to produce one or more phospholipids, unnaturally, at a concentration of, for example, 0.1 to 15.0 g/L, 0.5 g/L to 20 g/L, 0.75 g/L to 30 g/L, 1.0 g/L to 40 g/L, 1.25 g/L to 55.0 g/L, or about 50.0 g/L of the culture medium.

According to the subject methods, the phospholipid(s) can be retained in the cells of the microorganisms and/or secreted into the solid substrate and/or liquid medium in which the microbes are growing. In some embodiments, the phospholipid(s) can be recovered from the culture and purified according to known methods.

The inoculum with which fermentation is started according to the subject methods preferably comprises cells and/or propagules of the desired yeast, which can be prepared using any known fermentation method. In some embodiments, the propagules are spores.

In certain embodiments, production of the inoculum comprises seeding a nutrient medium (a nutrient medium that is not the customized nutrient medium) with cells of a yeast to produce a seed culture. This nutrient medium that is specific for inoculum production preferably comprises one or more components selected from ammonium sulfate, magnesium sulfate, di-potassium phosphate, monosodium phosphate, potassium phosphate, urea, yeast extract, glucose, and canola oil.

The seed culture is then cultivated at a temperate of, for example, about 28° C. The pH of seed culture cultivation preferably begins at about 6.0 and is stabilized at about 3.5. The DO levels are about 30% of saturation. The inoculum is then harvested upon the seed culture reaching a desired cell density. In certain embodiments, this takes from 3 to 9 days.

The inoculum is then used to inoculate the customized nutrient medium. The inoculant can remain pre-mixed with water and/or the nutrient medium in which it was cultivated, if desired. In certain embodiments, inoculating the customized nutrient medium with the inoculum can be performed by pipetting, pumping, pouring, sprinkling or spraying the inoculum into the vessel being used for fermentation.

In some embodiments, the method for cultivation may optionally comprise adding additional acids and/or antimicrobials into the substrate before and/or during cultivation.

In certain embodiments, after inoculating the customized nutrient medium, the yeast culture is cultivated for an amount of time ranging from about 2 days to about 10 days, or about 3 days to about 9 days. In one embodiment, phospholipid production can be observed in as little as 24 hours after the start of cultivation.

In one embodiment, the conditions favorable for production of phospholipids include specific temperature, dissolved oxygen (DO) and pH conditions.

In one embodiment, the favorable temperature is about 25 to 30° C., or about 26 to 28° C. In one embodiment, the favorable DO levels are about 20% to about 50% of saturation, or about 30%.

In one embodiment, the favorable pH levels are about 3.0 to about 7.0, or about 6.0. In certain embodiments, the cultivation pH begins at about 6.0 and is lowered to about 3.5 to 4.0 and stabilized. In certain embodiments, the pH naturally lowers during the course of cultivation. Thus, in some embodiments, the method can comprise simply stabilizing the pH upon reaching a pH of 3.5 to 4.0.

The methods for cultivation of microorganisms and production of microbial by-products can be performed in a batch process or a continuous/quasi-continuous process.

In one embodiment, all of the culture is removed upon completion of the cultivation (e.g., upon, for example, achieving a desired cell density, or concentration of phospholipid(s)). In this batch procedure, an entirely new batch is initiated after sterilization of the fermentation system.

In another embodiment, only a portion of the culture is removed at any one time. In this manner, a continuous or quasi-continuous system is created.

### Proposed Mechanism of Action

According to certain embodiments of the subject invention, inositol and inorganic phosphates are present in a culture medium of W. anomalus in separate forms; however, the presence of inositol stimulates production of polyol lipids, resulting in enhanced production of phospholipids. In some embodiments, the following mechanisms can explain how W. anomalus produces phospholipids, such as CL and/or precursors thereof.

Phytic acid, or phytate, is a unique natural substance found in plants, functioning as a principal phosphorus storage unit. When contained in plants, however, this form of phosphorous is not bioavailable to the digestive systems of non-ruminant animals that consume the plants.

W. anomalus, as a soil-inhabiting microbe, developed cascades of mechanisms for making phytate more bioavailable, which is beneficial for both yeast and plant species in the same habitat. The yeast produces phytase (myo-inositol hexakisphosphate phosphohydrolase), an enzyme that is able to catalyze hydrolysis of phytic acid, detaching phosphorus from an inositol molecule.

The removal of the phosphate group starts with a fully-phosphorylated phytic acid (IP₆), followed by penta- (IP₅), tetra- (IP₄), tri- (IP₃), di- and mono-esters of inositol in descending order of preference. This means that the phytase first hydrolyzes all of the available fully-phosphorylated phytic acid to penta-esters of inositol before hydrolyzing the latter to tetra-esters of inositol, and so on. A complete hydrolysis will ideally result in a myo-inositol and phosphate (plus amino acids, minerals and other nutrients linked to phytic acid).

After all of the phosphates are detached from the inositol molecule, W. anomalus initiates a reaction to convert inositol, a polyol, into a compound called polyol lipid, or liamocin. Liamocin is an extracellular form of carbon storage that the yeast can use exclusively for itself. This results in inhibition of other microorganisms in soil due to carbon sequestration and resulting carbon stress.

A biosynthetic pathway is proposed for liamocin production by a single non-acetylated 3,5-dihydroxydecanoate group. Malonyl-CoA derived from acetyl-CoA is condensed to form a C-4 carbon moiety by 3-ketoacyl synthase (KS), a condensing subunit present within a polyketide synthase (PKS) multicomplex. The nascent molecule undergoes a fully reductive cycle involving reduction via 3-ketoacyl-ACP reductase (KR), dehydration via dehydratase (DH) and a last reductive step via enoyl reductase.

Another malonyl-CoA is condensed to the molecule to produce a C-6 moiety that will undergo a fully reductive cycle. From this point on, two more condensations via KS followed by subsequent reductions with KR will yield a single 3,5-dihydroxydecanoate group still bound to the acyl carrier protein (ACP) of PKS. This pathway requires a 3-hydroxydecanoyl-ACP:CoA transacylase (PhaG) enzyme, encoded by a phaG gene to release the 3,5-dihydroxydecanoate group from ACP as a CoA derivative, before the incorporation of inositol.

Simultaneously with this pathway, production of an extracellular phospholipid, e.g., phosphatidylglycerol (PG) (General Formula 2), is observed. This is a byproduct of yeast survival and carbon conservation. PG is a known precursor for CL biosynthesis in eukaryotic cells. This molecule has similar functions to CL, but is more permeable through the membrane.

The pathway of PG production likely involves the following steps. Phosphatidic acid (PA) (General Formula 1) is synthesized in the endoplasmic reticulum (ER) and translocates to mitochondria in a process that is influenced by the ERMES (ER-mitochondria encounter structure) complex. Ups1/Mdm35p heterodimers transport PA from the outer membrane (OM) to the inner membrane (IM), potentially at contact sites. PA is converted to CDP-diacylglycerol (CDP-DAG) by Tam41p on the matrix-facing leaflet of the IM. CDP-DAG is used to generate phosphatidylglycerophosphate (PGP) by Pgs1p. PGP is dephosphorylated by Gep4p to produce PG. PG and another CDP-DAG are condensed to form un-remodeled CL by Crd1p. CL is de-acylated by Cldlp on the matrix-facing leaflet of the IM, forming monolysocardiolipin (MLCL) (General Formula 3).

Via an unknown mechanism, MLCL must flip to the IMS-facing leaflet of the IM or be transported to the OM to gain access to the transacylase Tazip, which regenerates CL. Multiple rounds of de-acylation/re-acylation result in remodeled CL, which is enriched in unsaturated acyl chains. CL achieves its final distribution on both leaflets of the IM and OM.

The presence of exogenous inositol downregulates biosynthesis of two phospholipids, phosphatidylcholine and phosphatidylinositol (General Formula 1), through transcriptional repression via an inositol-sensitive upstream activating sequence (UAS_{INO}). Pgs1p activity is also reduced in the presence of inositol. The inhibition of phosphatidylcholine and phosphatidylinositol trigger the yeast to produce more PGS1 mRNA, to produce increased amounts of PG, which is able to partially compensate for lack of phosphatidylcholine and phosphatidylinositol in cellular reactions.

Independent from its inositol-mediated regulation, Pgs1p activity is increased under conditions indicative of mitochondrial biogenesis; its mRNA abundance is highest when cells enter the stationary phase, and its activity is higher in the presence of non-fermentable carbon sources, such as, e.g., inositol. Importantly, PGS1 activity is upregulated by such conditions, resulting in increased amount of Pgs1p, which, however, is downregulated by phosphorylation in the presence of inositol.

Crd1p activity is similarly increased during stationary growth, in the presence of mtDNA, and in the presence of non-fermentable carbon sources, e.g., inositol, leading to increased CL levels. Therefore, a cell starts to require itself to produce more Pgs1p as a reaction to the overproduction of Crd1p. Because mRNA levels of PGS1 are upregulated, and the cell is living in an environment having increased Pgs1p activity, the cell starts to produce more and more Pgs1p due to phospholipid “starvation.” At the same time, the cell detects mitochondrial “toxicity” of inositol and begins converting inositol into polyol lipid by the mechanisms described above.

These reactions result in production of polyol lipids, and while inositol is converted into polyol lipid form, Pgs1p is de-phosphorylated, and Pgs1p activity increases rapidly. This leads to a burst of Pgs1p-catalyzed reactions and overproduction of phospholipids, and a resulting release of phospholipids, e.g., PG, outside the cell.

### Purification and Preparation of Microbe-Based Products

In certain embodiments, the subject invention provides microbe-based products, which can be used in a variety of settings including, for example, cosmetics and personal care products; human and animal health supplements; pharmaceuticals; oil and gas production; bioremediation and mining; waste disposal and treatment; and plant health and productivity (e.g., agriculture, horticulture, crops, pest control, forestry, turf management, and pastures).

One microbe-based product of the subject invention is simply a yeast culture comprising cells of a phospholipid-producing yeast, a nutrient medium, and a high concentration of a phospholipid. The phospholipid can be retained in the cells of the yeast and/or present as a secretion in the nutrient medium. The yeast culture can also comprise other metabolites produced by the yeast. The product of fermentation may be yeast culture can be harvested from the vessel and used directly, although, in preferred embodiments, the phospholipid growth by-products are extracted and, in certain embodiments, purified.

In a specific preferred embodiment, the composition comprises CL molecules, precursors thereof, and/or phospholipid molecules having similar structure and/or function thereto. All or a portion of the product can also be dried and later dissolved in water or another carrier.

In some embodiments, microbe-based product can comprise high concentrations of phospholipid(s), for example, about 10 ppm to about 10,000 ppm, about 100 ppm to about 5,000 ppm, about 200 to about 1,000 ppm, about 300 ppm to about 800 ppm, or about 500 ppm.

In some embodiments, the microbe-based product can comprise, for example, about 0.1 to 15.0 g/L, 0.5 g/L to 20 g/L, 0.75 g/L to 30 g/L, 1.0 g/L to 40 g/L, 1.25 g/L to 55.0 g/L, or about 50.0 g/L of the phospholipid(s).

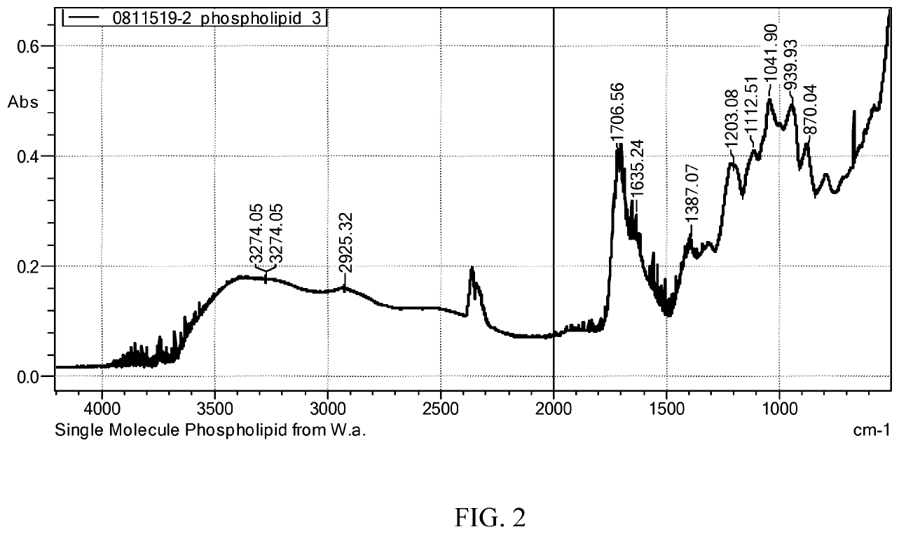

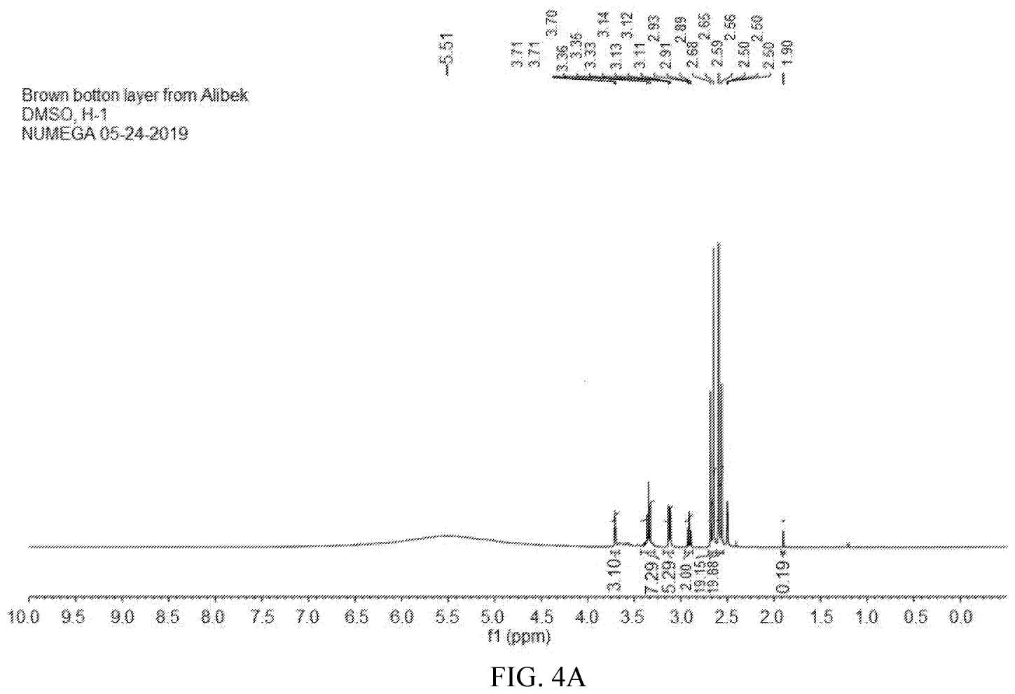

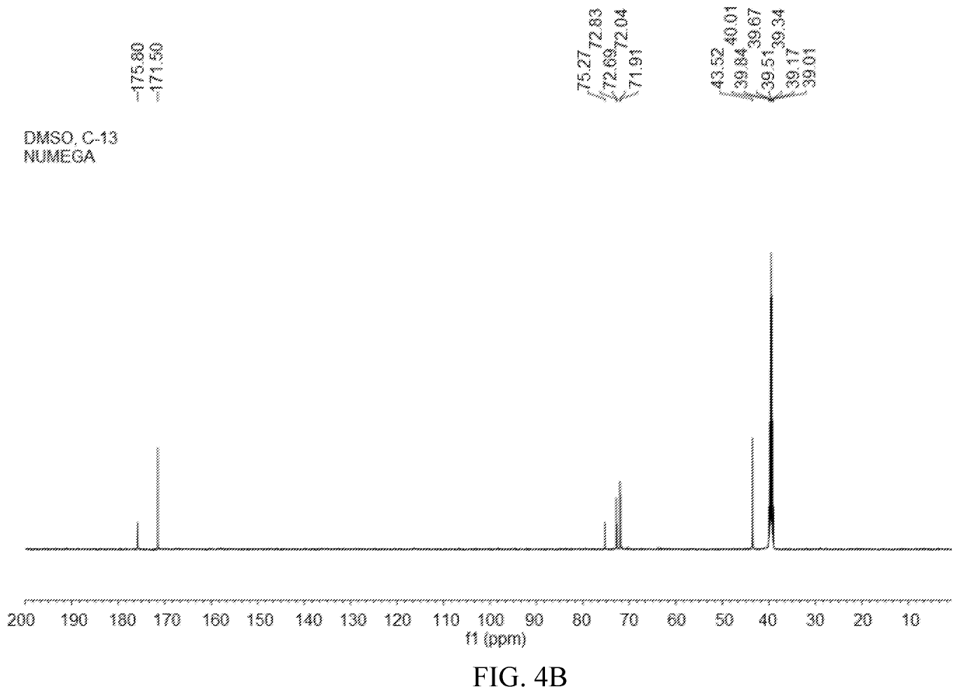

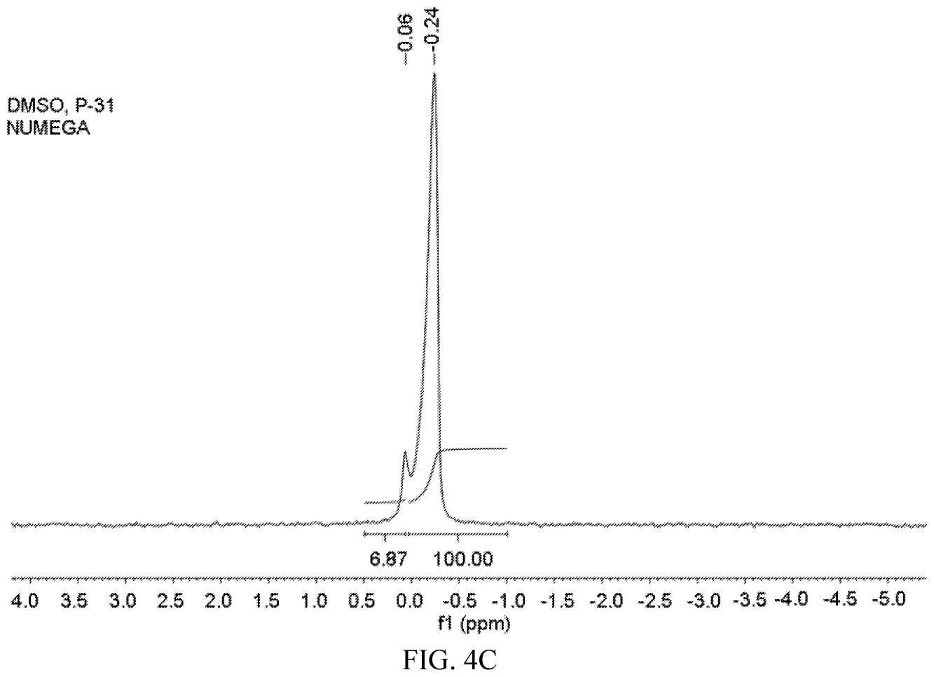

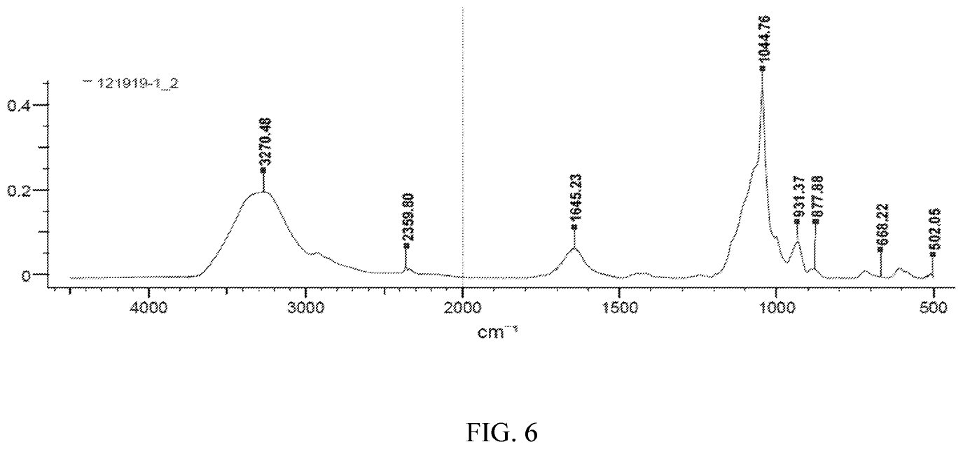

In some embodiments, the phospholipid is characterized and/or identified using known analytical methods. For example, in some embodiments, the phospholipid is characterized using Fourier-transform infrared spectroscopy (FTIR) analysis (FIG. 2, FIG. 6), NMR analysis (FIGS. 4A-4C), and/or mass spectrometry. In some embodiments, the phospholipid is characterized using observational analysis, for example, by observing the solubility of the compound in various solvents.

In certain embodiments, the compositions according to the subject invention can have advantages over, for example, purified microbial metabolites alone, due to, for example, the use of the entire culture. When producing yeasts, for example, the composition can comprise high concentrations of mannoprotein as a part of yeast cell wall's outer surface (mannoprotein is a highly effective bioemulsifier). Additionally, the compositions can comprise a variety of microbial metabolites (e.g., biosurfactants, enzymes, acids, solvents, and other) in the culture that may work in synergy with one another to achieve a desired effect.

Advantageously, in accordance with the subject invention, the microbe-based product may comprise the substrate in which the microbes were grown. In one embodiment, the composition may be, for example, at least 1%, 5%, 10%, 25%, 50%, 75%, or 100%, by weight, growth medium. The amount of biomass in the composition, by weight, may be, for example, anywhere from 0% to 100% inclusive of all percentages therebetween.

If present in the microbe-based product, the microorganisms may be in an active or inactive form. The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

In one embodiment, the composition does not comprise living microorganisms. In one embodiment, the composition does not comprise microorganisms, whether living or inactive.

In one embodiment, the compositions comprise one or more microbial growth by-products, wherein the growth by-product has been extracted from the culture and, optionally, purified. For example, in some embodiments, the yeast culture is mixed with ethyl acetate in a ratio of 1:1 for at least 40 to 100 hours, or 48 to 96 hours.

Afterwards, the mixture is centrifuged at 5,000 to 10,000×g for about 20 to 30 minutes, producing a cell pellet, a water phase comprising the phospholipid, a middle solid phase, and an ethyl acetate phase. The water phase is collected and evaporated to leave behind a brown mass. The brown mass comprises a purified phospholipid.

In some embodiments, the products include other microbial growth by-products, in addition to the phospholipids, including, for example, other biosurfactants, enzymes and/or metabolites.

In one embodiment, the composition comprises other biosurfactants. These other biosurfactants can be glycolipids and/or glycolipid-like biosurfactants, such as, for example, rhamnolipids (RLP), sophorolipids (SLP), mannosylerythritol lipids (MEL) and/or trehalose lipids. In one embodiment, the biosurfactants comprise lipopeptides and/or lipopeptide-like biosurfactants, such as, e.g., surfactin, iturin, fengycin, athrofactin, viscosin and/or lichenysin. In one embodiment, the biosurfactants comprise polymeric biosurfactants, such as, for example, emulsan, lipomanan, alasan, and/or liposan.

In some embodiments, the composition can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 0.1 gallon to 1,000 gallons or more. In certain embodiments the containers are 0.5 gallon, 2 gallons, 5 gallons, 25 gallons, or larger.

The microbe-based product can be removed from the container and transferred to the site of application via, for example, tanker, for immediate use.

Upon harvesting the microbe-based composition from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pesticides, and other ingredients specific for an intended use.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C. On the other hand, a biosurfactant composition can typically be stored at ambient temperatures.

### Methods of Use

The compositions of the subject invention can be used for a variety of purposes, including, for example, in the agriculture, oil and gas, cleaning product, pharmaceutical and supplement, human and animal health, and cosmetics industries.

Pharmaceutical and/or Health Supplement Product

In one embodiment, the compositions can be utilized in pharmaceutical and/or supplement products for enhancing human and animal health by, for example, improving mitochondrial function in humans and other animals. The pharmaceutical or health supplement can be formulated for oral administration, or any other mode of administration.

The compositions can be formulated into preparations in, for example, solid, semi-solid, liquid or inhalable forms, such as tablets, capsules, powders, granules, ointments, gels, lotions, solutions, suppositories, drops, patches, injections, inhalants, and aerosols.

In some embodiments, the composition can further comprise additional ingredients, such as, for example, one or more pharmaceutically-acceptable carriers and/or excipients, sources of energy, nutrients and/or other health-promoting compounds, flavorings, preservatives, prebiotics, pH adjusters, sweeteners and/or dyes. The term “pharmaceutically acceptable” as used herein means compatible with the other ingredients of a pharmaceutical, nutraceutical or food composition and not deleterious to the recipient thereof.

In some embodiments, the composition comprises, or is administered concurrently with, one or more additional health-promoting compounds for treating and/or preventing a certain disease, condition or disorder. “Health-promoting compounds” comprise any molecule or molecules that are meant to be administered to the digestive tract, blood and/or lymphatic circulation, as well as into tissues and organs, and ultimately reach a site in a subject's body where a positive impact on the subject's health can be effected. Non-limiting examples of health-promoting compounds include pharmaceuticals and/or nutritional supplements categorized as pain-relievers, antihistamines, antivirals, anticancer and/or chemotherapeutic compounds, antibiotics, antimicrobials, antiseizure compounds, anti-inflammatory compounds, antipsychotics, vaccines, statins, antidepressants, vitamins, minerals, nutrients, water and many others.

Carriers and/or excipients can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavorings, aromatisers, thickeners, coatings, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like.

In some cases, the carriers can be, for example, sterile aqueous or non-aqueous solutions, suspensions, and emulsions. Examples of non-aqueous solvents include, without limitation, propylene glycol, polyethylene glycol, vegetable oils, and organic esters. Aqueous carriers include, without limitation, water, alcohol, saline, and buffered solutions. Acceptable carriers also can include physiologically acceptable aqueous vehicles (e.g., physiological saline) or other known carriers appropriate to specific routes of administration. The use of carriers and/or excipients in the field of drugs and supplements is well known. Except for any conventional media or agent that is incompatible with the supplement composition or with, its use in the present compositions may be contemplated.

The pharmaceutical and/or health supplement formulation can be used for treating symptoms of mitochondrial deficiencies and/or secondary mitochondrial dysfunction caused by, for example, mitochondrial myopathy, Leigh syndrome, mitochondrial DNA depletion syndrome, Alzheimer's disease, muscular dystrophy, Lou Gehrig's disease, HIV, Bartonellosis, chronic fatigue syndrome, antiphospholipid syndrome, Barth syndrome, Parkinson's disease, non-alcoholic fatty liver disease and heart failure, Tangier disease, diabetes and/or cancer. Thus, methods are provided for treating and/or preventing such conditions and/or symptoms in a human or animal subject, wherein a therapeutically-effective amount of the composition is administered to the subject via oral, nasal, ocular, intravenous, intramuscular, topical, subcutaneous, anal, vaginal, and/or any other mode of administration.

As used herein, the term “therapeutically-effective amount,” is used to refer to an amount or dose of a compound or composition that, when administered to a subject, is capable of treating or improving a condition, disease, or disorder in a subject or that is capable of providing enhancement in health or function to an organ, tissue, or body system.

In certain specific embodiments, the composition can be used to treat and/or prevent a virus, such as influenza A, Herpes Simplex Virus (HSV)-1, HSV-2, Vesicular Stomatitis Virus (VSV), Ebolavirus, coronaviruses such as Severe Acute Respiratory Syndrome (SARS) coronavirus, Vaccinia Virus, Kaposi's Sarcoma-Associated Herpesvirus (KSHV).

In some embodiments, the composition can be applied to animal feed or water, or mixed with the feed or water, and used to prevent the spread of disease in livestock and aquaculture operations, reduce the need for antibiotic use in large quantities, as well as to provide supplemental proteins and other nutrients.

In certain specific embodiments, the pharmaceutical and/or supplemental composition can be used as an anti-aging product, wherein the composition can, for example, increase cell life, enhance youthful appearance of skin and hair, enhance functioning of organs, bones, tendons, joints and other tissues, enhance the immune system, enhance cognition, and overall, increase the lifespan or potential lifespan of a subject.

### Anti-Aging Cosmetic Product

In one embodiment, the compositions of the subject invention can serve as a replacement to costly synthetic cardiolipin and cardiolipin-like compounds currently used by the cosmetics industry. Thus, in some embodiments, the compositions can be formulated into a topical or injectable cosmetic product.

In certain embodiments, the cosmetic compositions can be used to treat and/or prevent a variety of skin conditions, including, for example, age spots, acne, scars, psoriasis, eczema, body odor, aging-related conditions (e.g., wrinkles, looseness, discoloration and dryness), and/or scalp conditions (e.g., dandruff, seborrheic dermatitis and hair loss).

In certain embodiments, the composition is applied directly to an area of the skin where such a condition exists. The composition can be applied to any external area of skin, including, for example, the skin of the face, ears, scalp, neck, back, shoulders, arms, hands, fingers, chest, torso, abdomen, underarms, feet, toes, buttocks, and legs.

In some embodiments, “applying” the composition can comprise leaving the composition on the area of skin, and/or rubbing it in so that the composition is absorbed into the area completely. In some embodiments, the composition can be applied to the skin for a therapeutically-effective amount of time and then rinsed or removed from the skin using, for example, water or a cloth. In some embodiments, the composition can be injected, e.g., subcutaneously and/or intradermally.

In yet another embodiment, the composition can be impregnated into a wound dressing and applied to the skin by covering the wound with the impregnated dressing according to standard dressing procedures.

In certain embodiments, the composition is applied from zero to ten times daily, preferably at least once per day or at least once every other day. In some embodiments, the composition is applied daily or every other day for an indefinite period of time, e.g., for at least one, two, three weeks, or longer, in order to achieve and/or maintain the treatment of the skin condition.

In one embodiment, the composition can be applied to the skin in liberal amounts, preferably to cover the entire area desired to be treated; however, only a thin coating should be needed to achieve a desired effect. In one embodiment, the composition is applied in an amount from about 0.001 to about 100 mg per cm² of skin, more typically from about 0.01 to about 20 mg/cm², or from about 0.1 to about 10 mg/cm². More or less may be used, however, depending upon the size of the area of skin to be treated.

Advantageously, in some embodiments, the cosmetic compositions can be used to heal and/or repair damaged skin, slow and/or reverse certain signs of aging, and/or treat a skin condition by increasing the cardiolipin levels of epithelial cells and/or enhancing skin cell mitochondrial function. In some embodiments, the composition promotes healing of damaged or wounded skin, and restores the skin's firmness, moisture and youthful appearance.

In certain embodiments, the cosmetic product can be, for example, a topical lotion, cream, gel, and/or face mask comprising the cardiolipin-like phospholipids produced according to the subject methods, and, optionally, live or inactive yeast cells and/or other growth by-products thereof.

In some embodiments, the cosmetic composition can further comprise a dermatologically-acceptable carrier or vehicle. As used herein, “dermatologically acceptable” means that a particular component is safe and non-toxic for application to a human integument (e.g., skin) at the levels employed. In one embodiment, the components of the composition are recognized as being Generally Regarded as Safe (GRAS).

The carrier or vehicle may include, for example, water; saline; physiological saline; ointments; creams; oil-water emulsions; water-in-oil emulsions; silicone-in-water emulsions; water-in-silicone emulsions; wax-in-water emulsions; water-oil-water triple emulsions; microemulsions; gels; vegetable oils; mineral oils; ester oils such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, preferably organomodified derivatives including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, carnauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 1% to about 99% by weight of the composition, from 10% to about 85%, from 25% to 75%, or from 50% to about 65%.

As used herein, the term “oil” includes silicone oils unless otherwise indicated. The emulsion may include an emulsifier, such as a nonionic, anionic or amphoteric surfactant, or a gallant, typically in an amount from about 0.001% to about 5% by weight.

In some embodiments, the composition can further comprise additional adjuvants and additives commonly included in skin care compositions, such as, for example, organic solvents, stabilizers, silicones, thickeners, softeners, sunscreens, moisturizers, fragrances or others described herein. The amounts of each ingredient, whether active or inactive, are those conventionally used in the cosmetic field to achieve their intended purpose, and typically range from about 0.0001% to about 25%, or from about 0.001% to about 20% of the composition, although the amounts may fall outside of these ranges. The nature of these ingredients and their amounts must be compatible with the production and function of the compositions of the disclosure.

In one embodiment, the composition may include additional skin actives, including but not limited to, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors, to name only a few.

In one embodiment, the composition may include additional anti-aging components, including, but not limited to, botanicals (e.g., Butea frondosa extract); phytol; phytonic acid; phospholipids other than those described herein; silicones; petrolatum; triglycerides; omega fatty acids; retinoids; hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof). When present, the additional anti-aging compounds can be included in amounts from about 0.0001% to about 5% by weight, more typically from about 0.01% to about 2.5% by weight, or from about 0.1% to about 1.0% by weight.

In one embodiment, the composition may include an exfoliating agent. Suitable exfoliating agents include, for example, alpha-hydroxy acids, beta-hydroxy acids, oxa-acids, oxadiacids, and their derivatives, such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

In one embodiment, the composition may comprise one or more antioxidants. Suitable antioxidants include, for example, compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. Antioxidants may comprise, individually or collectively, from about 0.001% to about 10% (w/w), or from about 0.01% to about 5% (w/w) of the total weight of the composition.

Non-biological surfactants can also be added to the formulation. Examples of surfactants include, but are not limited to, alkyl sulfates, alkyl ether sulfates (e.g., sodium/ammonium lauryl sulfates and sodium/ammonium laureth sulfates), amphoterics (e.g., amphoacetates and amphopropionates), sulfosuccinates, alkyl polyglucosides, betaines (e.g., cocamidopropul betaine (CAPB)), sultaines, sacrosinates, isethionates, taurates, ethoxylated sorbitan esters, alkanolamides and amino-acid based surfactants.

Viscosity modifiers can also be added to the compositions, including, for example, cocamide DEA, oleamide DEA, sodium chloride, cellulosic polymers, polyacrylates, ethoxylated esters, alcohol, glycols, xylene sulfonates, polysorbate 20, alkanolamides, and cellulose derivatives (e.g., hydroxypropyl methylcellulose and hydroxyethyl cellulose).

Polymers can also be added, including, for example, xanthan gum, guar gum, polyquaternium-10, PEG-120 methyl glucose dioleate, PEG-150 distearate, PEG-150 polyglyceryl-2 tristearate and PEG-150 pentaerythrityl tetrastearate

A sunscreen or combination of sunscreens may be included to protect the skin from both UVA and UVB rays. Among the sunscreens that can be employed in the present compositions are avobenzone, cinnamic acid derivatives (such as octylmethoxy cinnamate), octyl salicylate, oxybenzone, octocrylene, titanium dioxide, zinc oxide, or any mixtures thereof. The sunscreen may be present from about 1 wt % to about 30 wt % of the total weight of the composition.

The composition may optionally comprise other components, additives or adjuvants known to those skilled in the art including, but not limited to: skin penetration enhancers; emollients (e.g., isopropyl myristate, petrolatum, volatile or non-volatile silicones oils, such as methicone and dimethicone, ester oils, mineral oils, and fatty acid esters); humectants (e.g., glycerin, hexylene glycol, caprylyl glycol); skin plumpers (e.g., palmitoyl oligopeptide, collagen, collagen and/or glycosaminoglycan (GAG) enhancing agents); anti-inflammatory agents (e.g., Aloe vera, bioflavonoids, diclofenac, salicylic acid); chelating agents (e.g., EDTA or a salt thereof, such as disodium EDTA); vitamins (e.g., tocopherol and ascorbic acid); vitamin derivatives (e.g., ascorbyl monopalmitate, tocopheryl acetate, Vitamin E palmitate); thickeners (e.g., hydroxyalkyl cellulose, carboxymethylcellulose, carbombers, and vegetable gums, such as xanthan gum); gelling agents (e.g., ester-terminated polyester amides); structuring agents; proteins (e.g., lactoferrin); immune modulators (e.g., corticosteroids and non-steroidal immune modulators).

Other components that may be included are film formers, moisturizers, minerals, viscosity and/or rheology modifiers, insect repellents, skin cooling compounds, skin protectants, lubricants, preservatives, pearls, chromalites, micas, conditioners, anti-allergenics, antimicrobials (e.g., antifungals, antivirals, antibacterials), antiseptics, pharmaceutical agents, photostabilizing agents, surface smoothers, optical diffusers, and exfoliation promoters. Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute from about 0.01% to about 20% of the total weight of the composition.

The composition can include pH adjusters (e.g., citric acid, ethanolamine, sodium hydroxide, etc.) to be formulated within a wide range of pH levels. In one embodiment, the pH of the composition ranges from 1.0 to 13.0. In some embodiments, the pH of the composition ranges from 2.0 to 12.0. Other pH ranges suitable for the subject composition include from 3.5 to 7.0, or from 7.0 to 10.5. Suitable pH adjusters such as sodium hydroxide, citric acid and triethanolamine may be added to bring the pH within the desired range.

The composition may be formulated as a suspension, emulsion, hydrogel, multiphase solution, vesicular dispersion or in any other known form of topical skin composition.

In certain embodiments, the composition may be formulated so that it can be applied, for example, via pen, tube, bottle, brush, stick, sponge, cotton swab, towelette (wipe), sprayer, dropper, hand or finger.

The composition may be formulated in a variety of product forms, such as, for example, a lotion, cream, serum, mask, spray, aerosol, liquid cake, ointment, essence, gel, paste, patch, pencil, powder, towelette, soap, shampoo, conditioner, stick, foam, mousse, elixir or concentrate.

In one embodiment, the composition can be incorporated into a dressing or bandage that may be applied, attached or coupled to one or more layers of the skin or tissue of the subject. For example, the composition may be applied to a dressing, which can then be placed over the area of skin being treated.

### Local Production of Microbe-Based Products

In certain embodiments of the subject invention, a microbe growth facility produces fresh, high-density microorganisms and/or microbial growth by-products of interest on a desired scale. The microbe growth facility may be located at or near the site of application (e.g., a cosmetics factory). The facility produces high-density microbe-based compositions in batch, quasi-continuous, or continuous cultivation.

The microbe growth facilities of the subject invention can be located at the location where the microbe-based product will be used. For example, the microbe growth facility may be less than 300, 250, 200, 150, 100, 75, 50, 25, 15, 10, 5, 3, or 1 mile from the location of use.

Because the microbe-based product can be generated locally, without resort to the microorganism stabilization, preservation, storage and transportation processes of conventional microbial production, a much higher density of microorganisms and/or metabolites can be generated, thereby requiring a smaller volume of the microbe-based product for use in the on-site application, or which allows much higher density applications where necessary to achieve the desired efficacy. Local generation of the microbe-based product also facilitates the inclusion of the growth medium in the product. The medium can contain agents produced during the fermentation that are particularly well-suited for local use.

Locally-produced high density, robust cultures of microbes and/or their growth by-products are more effective in the field than those that have remained in the supply chain for some time. Reduced transportation times allow for the production and delivery of fresh batches of microbes and/or their metabolites at the time and volume as required by local demand.

The microbe growth facilities of the subject invention produce fresh, microbe-based compositions, comprising the microbes themselves, microbial metabolites, and/or other components of the medium in which the microbes are grown. If desired, the compositions can have a high density of vegetative cells or propagules, or a mixture of vegetative cells and propagules.

In one embodiment, the microbe growth facility is located on, or near, a site where the microbe-based products will be used, for example, within 300 miles, 200 miles, or even within 100 miles. Advantageously, this allows for the compositions to be tailored for use at a specified location. The formula and potency of microbe-based compositions can be customized for a specific application and in accordance with the local conditions at the time of application.

Advantageously, distributed microbe growth facilities provide a solution to the current problem of relying on far-flung industrial-sized producers whose product quality suffers due to upstream processing delays, supply chain bottlenecks, improper storage, and other contingencies that inhibit the timely delivery and application of, for example, a viable, high cell-count product and/or metabolite concentration, and the associated medium in which the cells are originally grown.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention.

### Example 1—Production of W. anomalus Inoculum

An inoculum of Wickerhamomyces anomalus is cultivated using submerged cultivation in a culture medium comprising:
Ammonium sulfate—2.5 g/L
Magnesium sulfate—0.2 g/L
Di-potassium phosphate—0.2 g/L
Potassium phosphate—1 g/L
Monosodium phosphate—3 g/L
Urea—1 g/L
Yeast extract—1 g/L
Glucose—20 g/L
Canola oil—30 ml/L.

Initially, pH is set to 6.0 and temperature is set to 28° C. pH is allowed to decrease to about 3.5, where it is stabilized. DO is 30% of saturation. Cardiolipin-like phospholipids are produced within 3 days, with a maximum concentration obtained at day 9.

### Example 2—Enhanced Production of Phospholipids in Customized Nutrient Medium Using W. anomalus

An inoculum as produced in Example 1 above is used to inoculate a submerged fermentation reactor containing a culture medium comprising:
Ammonium sulfate—2.5 g/L
Magnesium sulfate—0.2 g/L
Di-potassium phosphate—0.2 g/L
Potassium phosphate—1 g/L
Monosodium phosphate—3 g/L
Urea—1 g/L
Yeast extract—0.5 g/L
Glucose—20 g/L
Soybean and/or sunflower oil—50 mi/L.
Trace elements—0.5 ml/L
Inositol—10 g/L
Biotin—0.1 g/L
Acetyl L-carnitine—0.2 g/L
Alpha-lipoic acid—0.5 g/L

Initially, pH is set to 6.0 and temperature is set to 28° C. pH is allowed to decrease to about 3.5, where it is stabilized. DO is 30% of saturation. Extracellular phospholipids are produced within 3 days, with a maximum concentration obtained by day 9. The total concentration of phospholipids produced from an 8 L reactor is 4 g (0.5 g/L).

### Example 3—Purification of Phospholipids from Yeast Culture

When the nutrient medium utilized in Examples 1 and 2 supra turns a pink color, this signals the time for harvesting of the culture. The entire yeast culture is harvested and mixed with ethyl acetate at a ratio of 1:1 by volume for at least 40 to 100 hours, preferably about 48 hours to 96 hours.

After mixing, the culture and ethyl acetate mixture is centrifuged at 8,000×g for 30 minutes to produce a cell pellet, a water phase, a middle solid phase and a top ethyl acetate layer.

The water phase is collected and evaporated at 55 to 65° C. until the resulting product is a viscous, concentrated brown mass. The mass comprises purified phospholipids at a purity of 90% by weight or greater.

### Example 4—Analysis of Phospholipids Produced by W. anomalus

The phospholipid was treated according to Table 1 to test for solubility in different solvents. It is important that the sample was soluble in water, given that only a few phospholipids are soluble in water, such as phosphatidylglycerol.

TABLE 1
Observational Solubility Analysis of Phospholipid

| Solvent | Solubility in Solvent |
|---|---|
| H₂O | Highly soluble - almost entirely dissolved |
| Isopropyl alcohol | Poor - solid materials collected at |
| | bottom of liquid |
| MeOH | Poor - solids and cloudy materials |
| | collected at bottom of liquid |
| Hexanes | Poor - solid materials collected at |
| | bottom of liquid |
| CH₂Cl₂ | Poor - solid materials collected at |
| | bottom of liquid |
| 60:40 Acetonitrile:Water | Medium - cloudy at first, but a fair amount |
| Formic | dissolves, leaving only some solid material |
| | at the bottom |
| 70:24:6 | Medium - cloudy at first, but a fair amount |
| MeOH:Acetonitrile:Water | dissolves, leaving some solid material at |
| Formic | the bottom (a greater amount than |
| | previous solvent) |
| 65:15:2 | Poor - most of the sample floated to the |
| Chloroform:MeOH:Water | top of the liquid |

Fourier-transform infrared spectroscopy (FT-IR) analyses were also performed on purified phospholipid produced according to embodiments of the subject invention. The results are depicted in FIG. 2 and FIG. 6. The FT-IR analyses shows that the phospholipid contained a C═O carbonyl-ester group and an —OH.

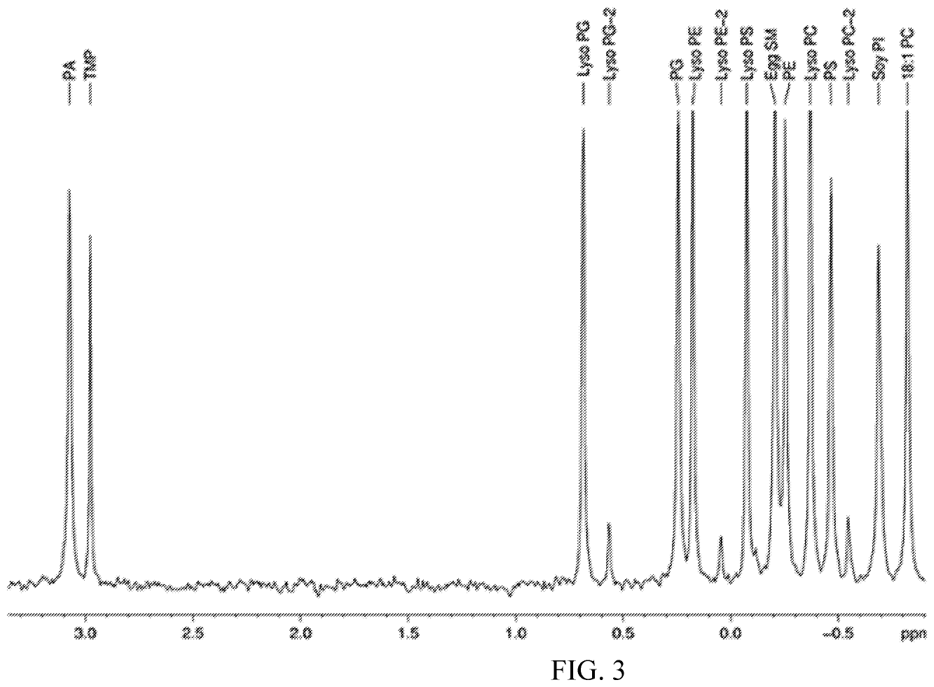

P-31 NMR analysis was performed on a variety of phospholipids for comparison with the subject phospholipids. FIG. 3.

Various NMR analyses were also performed on the purified phospholipid of the subject invention. The results are depicted in FIGS. 4A-4C. The H-1 NMR (FIG. 4A) showed that the phospholipid contained a —CH2- functional group. The P-31 NMR (FIG. 4C) confirmed the presence of phosphorous species, and based on the chemical shift, suggested that the molecule was a phosphatidylglycerol or phosphatidylethanolamine. There was no evidence of phosphate salts in the sample.

### Example 5—Mass Spectrometry Analysis of Phospholipids Produced by W. anomalus

Mass spectrometry analysis was performed to compare CL from bovine cells and the phospholipids produced by W. anomalus. Samples were tested on a Shimadzu NexeraX2 UHPLC with a Shimadzu 2040 LCMS. Samples were dissolved into the starting mobile phase (50% Solvent D, 50% Solvent A) with a dilution factor of 2.

The samples were filtered through 0.22 um PVDF filter to remove particulate and injected at 10 uL and separated across a Sigma Aldrich Ascentis Express C18 Column (150 mm×2.1 mm×2.7 um). Solvent D: 90% Acetonitrile, 10% Water; Solvent A: 90% 2-Propanol, 10% Water. The pump flow rate was 0.4 mL/min.

The MS peaks for the phospholipid were as follows:
724 m/z, 727 m/z, 725 m/z—a+136 ion and b+136 ion from [M-H]— ion of (14:0/14:0)(14:0/14:0)-CL at m/z 1239 or d2-(14:0/14:0)(14:0/14:0)-CL at m/z 1241
591 m/z, 592 m/z—a and b ions of [M-H]— ion of (14:0/14:0)(14:0/14:0)-CL at m/z 1239 or d2-(14:0/14:0)(14:0/14:0)-CL at m/z 1241
647—a+56 ion and b+56 ion of [M-H]— ion of (14:0/14:0)(14:0/14:0)-CL at m/z 1239 or d2-(14:0/14:0)(14:0/14:0)-CL at m/z 1241
671, 672 m/z, 673 m/z, 675 m/z—[M-2H]2- ion of (16:0/16:1)(16:0/16:1)-CL
1347 m/z—[M-H]— ion of (16:0/16:1)(16:0/16:1)-CL
673 m/z, 729 m/z, 809 m/z—(16:0/18:1)(16:0/18:1)-CL

Other possible peaks indicating possibility of cardiolipin, resulting from molecules breaking into different ions, were (m/z): 227, 253, 255, 279, 281, 363, 389, 391, 409, 417, 435, 555, 569, 582, 619, 645, 647, 686, 699, 701, 728, 1093, 1240, 1448, and 1456.

### Example 6—Effects of Phospholipid Treatment on Drosophila Lifespan

Fruit flies were fed with a phospholipid according to embodiments of the subject invention to observe the effects on lifespan.

A nutrient medium comprising agar (15 g/L), molasses (130 g/L), cornmeal (100 g/L), dextrose (50 g/L), and casein peptone type M (20 g/L) was autoclaved at 121° C. for 15 minutes. Afterwards, the nutrient medium was allowed to cool to 60° C., at which time 10 g/L of the phospholipid was added to the medium for use in feeding Experimental group flies. The phospholipid was not added to the nutrient medium fed to Control group flies.

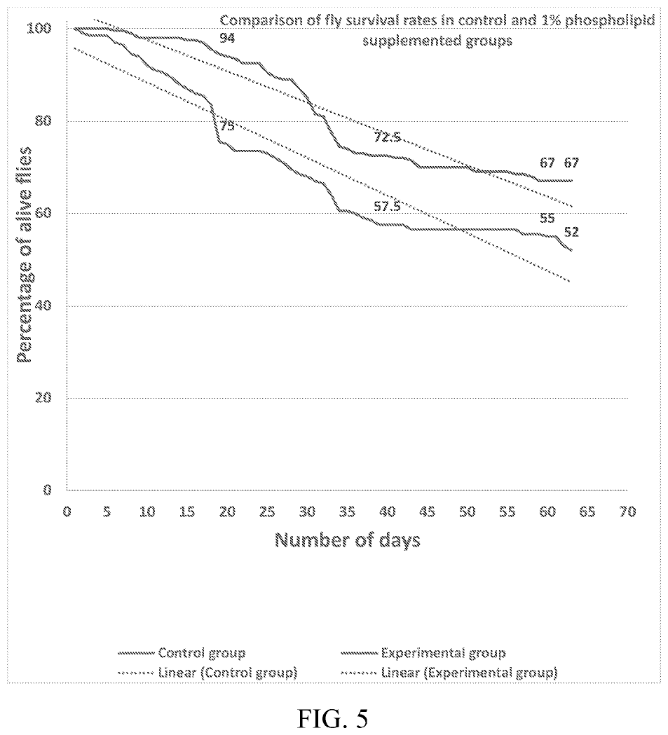

Approximately 20 ml of the nutrient medium was placed into individual vials. The vials were inoculated with fruit flies and the survival rate for each group was measured over the course of about 60 to 65 days. As shown in FIG. 5, the Experimental group exhibited 67% survival rate after about 63 days, while the Control group exhibited a 52% survival rate after the same time period.

### REFERENCES

Christie, W. W., “Cardiolipin (Diphosphatidylglycerol)”, The LipidWeb, updated Oct. 15, 2018, http://www.lipidhome.co.uk/lipids/complex/dpg/index.htm. (“The LipidWeb” 2018).
Pangborn M. A New Serologically Active Phospholipid from Beef Heart. Experimental Biology and Medicine. 1941; 48(2):484-486.
Pangborn M. Isolation and Purification of a Serologically Active Phospholipid From Beef Heart. The Journal of Biological Chemistry. 1942; (143):247-256.
Oemer G, Lackner K, Muigg K, Krumschnabel G, Watschinger K, Sailer S et al. Molecular structural diversity of mitochondrial cardiolipins. Proceedings of the National Academy of Sciences. 2018; 115(16):4158-4163.
Pizzuto M, Lonez C, Baroja-Mazo A, Martinez-Banaclocha H, Tourlomousis P, Gangloff M et al. Saturation of acyl chains converts cardiolipin from an antagonist to an activator of Toll-like receptor-4. Cellular and Molecular Life Sciences. 2019; 76(18):3679-3680.
Houtkooper R, Rodenburg R, Thiels C, Lenthe H, Stet F, Poll-The B et al. Cardiolipin and monolysocardiolipin analysis in fibroblasts, lymphocytes, and tissues using high-performance liquid chromatography-mass spectrometry as a diagnostic test for Barth syndrome. Analytical Biochemistry. 2009; 387(2):230-237.
Zhou Y, Peisker H, Dormann P. Molecular species composition of plant cardiolipin determined by liquid chromatography mass spectrometry. Journal of Lipid Research. 2016; 57(7):1308-1321.
Paradies G, Paradies V, De Benedictis V, Ruggiero F, Petrosillo G. Functional role of cardiolipin in mitochondrial bioenergetics. Biochimica et Biophysica Acta (BBA)—Bioenergetics. 2014; 1837(4):408-417.
Ye C, Lou W, Li Y, Chatzispyrou I, Hüttemann M, Lee I et al. Deletion of the Cardiolipin-specific Phospholipase Cld1 Rescues Growth and Life Span Defects in the Tafazzin Mutant. Journal of Biological Chemistry. 2013; 289(6):3114-3125.
Mileykovskaya E, Dowhan W. Cardiolipin-dependent formation of mitochondrial respiratory supercomplexes. Chemistry and Physics of Lipids. 2014; 179:42-48.
Ren M, Phoon C, Schlame M. Metabolism and function of mitochondrial cardiolipin. Progress in Lipid Research. 2014; 55:1-16.
Maguire J, Tyurina Y, Mohammadyani D, Kapralov A, Anthonymuthu T, Qu F et al. Known unknowns of cardiolipin signaling: The best is yet to come. Biochimica et Biophysica Acta (BBA)—Molecular and Cell Biology of Lipids. 2017; 1862(1):8-24.
Kiebish M, Han X, Cheng H, Chuang J, Seyfried T. Cardiolipin and electron transport chain abnormalities in mouse brain tumor mitochondria: lipidomic evidence supporting the Warburg theory of cancer. Journal of Lipid Research. 2008; 49(12):2545-2556.
Fry M, Green D. Cardiolipin requirement by cytochrome oxidase and the catalytic role of phospholipid. Biochemical and Biophysical Research Communications. 1980; 93(4):1238-1246.
Fry M, Green D. Cardiolipin requirement for electron transfer in complex I and III of the mitochondrial respiratory chain. The Journal of Biological Chemistry. 1981; 256(4):1874-1880.
Schug Z, Gottlieb E. Cardiolipin acts as a mitochondrial signalling platform to launch apoptosis. Biochimica et Biophysica Acta (BBA)—Biomembranes. 2009; 1788(10):2022-2031.
Gonzalvez F, D'Aurelio M, Boutant M, Moustapha A, Puech J, Landes T et al. Barth syndrome: Cellular compensation of mitochondrial dysfunction and apoptosis inhibition due to changes in cardiolipin remodeling linked to tafazzin (TAZ) gene mutation. Biochimica et Biophysica Acta (BBA)—Molecular Basis of Disease. 2013; 1832(8):1194-1206.
Sapandowski A, Stope M, Evert K, Evert M, Zimmermann U, Peter D et al. Cardiolipin composition correlates with prostate cancer cell proliferation. Molecular and Cellular Biochemistry. 2015; 410(1-2):175-185.
Lee H, Mayette J, Rapoport S, Bazinet R. Selective remodeling of cardiolipin fatty acids in the aged rat heart. Lipids in Health and Disease. 2006; 5(1):2.
Petrosillo G, Matera M, Casanova G, Ruggiero F, Paradies G. Mitochondrial dysfunction in rat brain with agingInvolvement of complex I, reactive oxygen species and cardiolipin. Neurochemistry International. 2008; 53(5):126-131.
Paradies G, Ruggiero F, Petrosillo G, Quagliariello E. Age-dependent decline in the cytochrome c oxidase activity in rat heart mitochondria: role of cardiolipin. FEBS Letters. 1997; 406(1-2):136-138.
Vorbeck M, Martin A, Long J, Smith J, Orr R. Aging-dependent modification of lipid composition and lipid structural order parameter of hepatic mitochondria. Archives of Biochemistry and Biophysics. 1982; 217(1):351-361.
Gómez L, Monette J, Chavez J, Maier C, Hagen T. Supercomplexes of the mitochondrial electron transport chain decline in the aging rat heart. Archives of Biochemistry and Biophysics. 2009; 490(1):30-35.
Gömez L, Hagen T. Age-related decline in mitochondrial bioenergetics: Does supercomplex destabilization determine lower oxidative capacity and higher superoxide production?. Seminars in Cell & Developmental Biology. 2012; 23(7):758-767.
Hagen T, Liu J, Lykkesfeldt J, Wehr C, Ingersoll R, Vinarsky V et al. Feeding acetyl-L-carnitine and lipoic acid to old rats significantly improves metabolic function while decreasing oxidative stress. Proceedings of the National Academy of Sciences. 2002; 99(4):1870-1875.
Paradies G, Petrosillo G, Pistolese M, Di Venosa N, Federici A, Ruggiero F. Decrease in Mitochondrial Complex I Activity in Ischemic/Reperfused Rat Heart. Circulation Research. 2004; 94(1):53-59.
Widlansky M, Wang J, Shenouda S, Hagen T, Smith A, Kizhakekuttu T et al. Altered mitochondrial membrane potential, mass, and morphology in the mononuclear cells of humans with type 2 diabetes. Translational Research. 2010; 156(1):15-25.
Park J, Davis R, Sue C. Mitochondrial Dysfunction in Parkinson's Disease: New Mechanistic Insights and Therapeutic Perspectives. Current Neurology and Neuroscience Reports. 2018; 18(5):21.
Chen S, He Q, Greenberg M. Loss of tafazzin in yeast leads to increased oxidative stress during respiratory growth. Molecular Microbiology. 2008; 68(4):1061-1072.
Baile M, Sathappa M, Lu Y, Pryce E, Whited K, McCaffery J et al. Unremodeled and Remodeled Cardiolipin Are Functionally Indistinguishable in Yeast. Journal of Biological Chemistry. 2013; 289(3): 1768-1778.
Numata M, Chu H, Dakhama A, Voelker D. Pulmonary surfactant phosphatidylglycerol inhibits respiratory syncytial virus-induced inflammation and infection. Proceedings of the National Academy of Sciences. 2009; 107(1):320-325.
Numata M, Kandasamy P, Voelker D. Anionic pulmonary surfactant lipid regulation of innate immunity. Expert Review of Respiratory Medicine. 2012; 6(3):243-246.
Numata M, Kandasamy P, Nagashima Y, Posey J, Hartshorn K, Woodland D et al. Phosphatidylglycerol Suppresses Influenza A Virus Infection. American Journal of Respiratory Cell and Molecular Biology. 2012; 46(4):479-487.
Kuronuma K, Mitsuzawa H, Takeda K, Nishitani C, Chan E, Kuroki Y et al. Anionic Pulmonary Surfactant Phospholipids Inhibit Inflammatory Responses from Alveolar Macrophages and U937 Cells by Binding the Lipopolysaccharide-interacting Proteins CD14 and MD-2. Journal of Biological Chemistry. 2009; 284(38):25488-25500.
Lester S, Li K. Toll-Like Receptors in Antiviral Innate Immunity. Journal of Molecular Biology. 2014; 426(6): 1246-1264.
Chen W, Chao Y, Chang W, Chan J, Hsu Y. Phosphatidylglycerol Incorporates into Cardiolipin to Improve Mitochondrial Activity and Inhibits Inflammation. Scientific Reports. 2018; 8, Article number 4919.

1. A method for producing a phospholipid, the method comprising:
inoculating a nutrient medium with an inoculum of a Wickerhamomyces anomalus yeast to produce a yeast culture comprising yeast cells and medium; and
cultivating the yeast culture for 3 to 9 days under conditions favorable for the production of the phospholipid, said phospholipid having a structure according to: 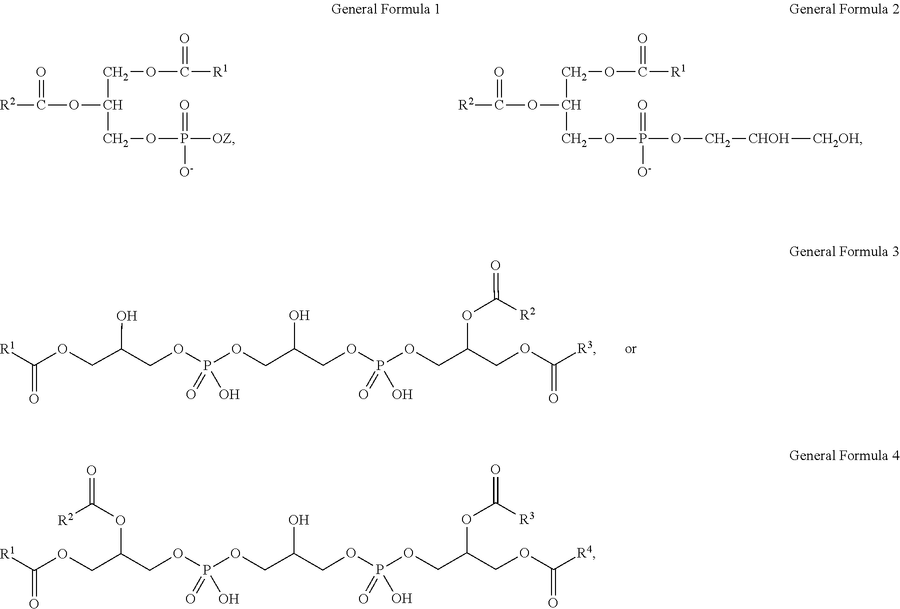
where Z is H, a serine group, choline group, ethanolamine group, inositol group, or glycerol group, and
where R1-R4 are the same or different fatty acid side chains having between 14 and 22 carbon atoms and between 0 and 6 double-bonded carbon atoms.

2. (canceled)

3. The method of claim 1, wherein the phospholipid is a phosphatidylglycerol having a structure according to General Formula 1, and wherein Z is a glycerol group.

4. The method of claim 1, wherein the phospholipid is a cardiolipin having a structure according to General Formula 1.

5. The method of claim 4, wherein R1, R2, R3 and R4 are linoleoyl groups.

6. (canceled)

7. The method of claim 1, wherein the favorable conditions include dissolved oxygen measurement of about 20% to about 50% of saturation.

8. The method of claim 1, wherein the favorable conditions include cultivation pH of about 3.0 to about 7.0.

9. The method of claim 1, wherein the nutrient medium comprises one or more inorganic salts; one or more sources of nitrogen; one or more sources of vitamins, proteins, growth factors, and/or trace nutrients; one or more sources of fatty acids; and one or more sources of carbon.

10. The method of claim 9, wherein the medium comprises one or more inorganic salts selected from ammonium sulfate, magnesium sulfate, di-potassium phosphate, monosodium phosphate, and potassium phosphate.

11. The method of claim 9, wherein the medium comprises one or more sources of nitrogen selected from potassium nitrate, ammonium nitrate ammonium sulfate, ammonium phosphate, ammonia, urea, and ammonium chloride.

12. The method of claim 9, wherein the medium comprises yeast extract.

13. The method of claim 9, wherein the medium comprises one or more sources of fatty acids selected from canola oil, sunflower oil, and soybean oil.

14-15. (canceled)

16. The method of claim 9, wherein the nutrient medium comprises ammonium sulfate, magnesium sulfate, di-potassium phosphate, monosodium phosphate, potassium phosphate, biotin, acetyl L-carnitine, alpha-lipoic acid, inositol, urea, yeast extract, glucose, soybean and/or sunflower oil, and trace elements.

17. The method of claim 9, wherein the nutrient medium comprises 5 g/L to 20 g/L inositol.

18-19. (canceled)

20. The method of claim 1, further comprising purifying the phospholipid from the yeast culture.

21. The method of claim 20, wherein purification comprises mixing the yeast culture with ethyl acetate at a ratio of 1:1 by volume to form a mixture;
mixing the mixture for about 40 to 100 hours, followed by centrifuging the mixture for 20 to 30 minutes to produce a cell pellet layer, a water layer, a middle solid layer and an ethyl acetate layer;
collecting the water layer; and
evaporating the water layer to produce a viscous brown mass comprising purified phospholipids.

22-24. (canceled)

25. A yeast culture comprising cells of a phospholipid-producing yeast, a nutrient medium, and a high concentration of a phospholipid, wherein the yeast is Wickerhamomyces anomalus, and wherein the phospholipid is retained in the cells of the yeast and/or is present as an extracellular secretion in the nutrient medium; wherein the high concentration of phospholipid is 0.1 g/L to 55.0 g/L of the medium.

26. (canceled)

27. The yeast culture of claim 25, wherein the phospholipid has a structure according to 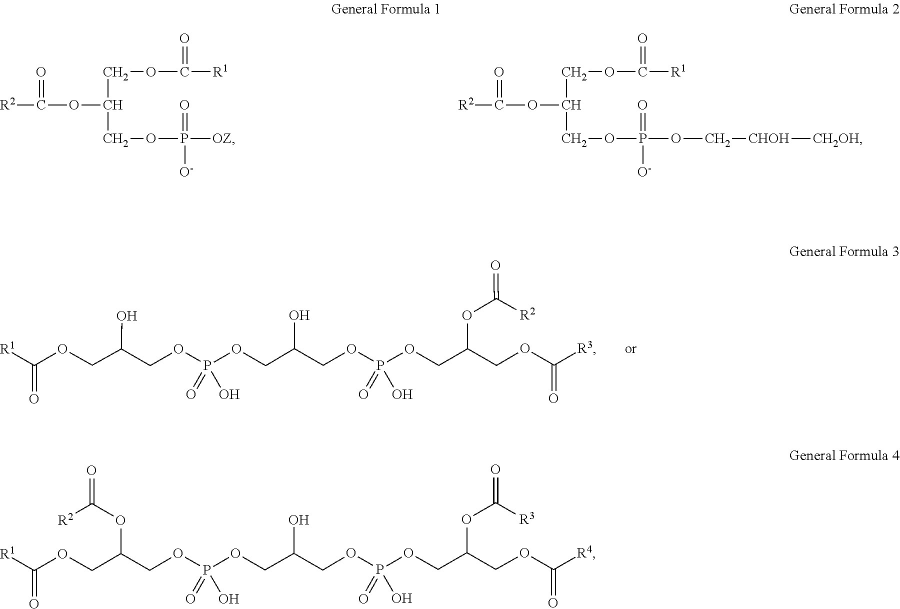
where Z is H, a serine group, choline group, ethanolamine group, inositol group, or glycerol group, and
where R1-R4 are the same or different fatty acid side chains having between 14 and 22 carbon atoms and between 0 and 6 double-bonded carbon atoms.

28-29. (canceled)

30. An anti-aging composition for reversing and/or slowing a sign of aging, the composition comprising a phospholipid molecule having a structure according to: 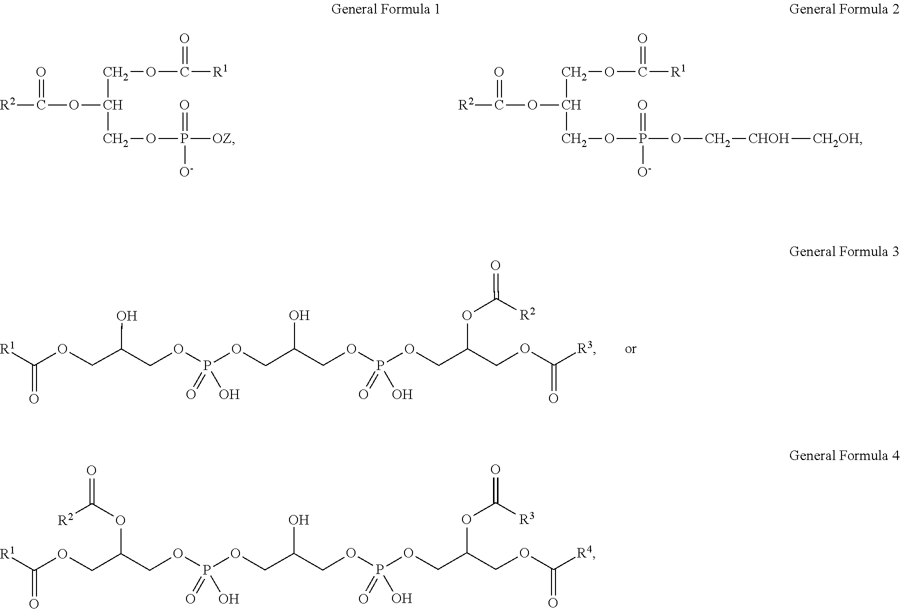
where Z is H, a serine group, choline group, ethanolamine group, inositol group, or glycerol group, and
where R1-R4 are the same or different fatty acid side chains having between 14 and 22 carbon atoms and between 0 and 6 double-bonded carbon atoms, and
wherein the phospholipid molecule was produced by cultivating a Wickerhamomyces anomalus yeast.

31. (canceled)

32. The anti-aging composition according to claim 30, formulated as a cosmetic composition, wherein the composition further comprises a dermatologically-acceptable carrier and, optionally, one or more additional skin-active agents.
